(19) United States
(12) Patent Application Publication
(10) Pub. No.: US 2005/0009203 A1
(43) Pub. Date: Jan. 13, 2005

(54) Multi-drug testing device and method

(76) Inventor: Johnson N.S. Wong, Rolling Hills, CA (US)

Correspondence Address: FULBRIGHT & JAWORKI L.L.P.;Robert Berliner, Twenty-Ninth Floor, 865 South Figueroa, Los Angeles, CA, 90017-2571, US

(21) Appl. No.: 10/619,158

(22) Filed: Jul. 11, 2003

(51) Int. Cl. G01N 33/543

(52) U.S. Cl. 436/518

(57) ABSTRACT

Drugs of abuse test strips are retained on the underside of a closure cap for a fluid sample container. Each strip has a bottom end defining a sample receiving portion and an indicator spaced longitudinally therefrom to visually indicate the presence of a selected drug of abuse. The closure cap is formed to seal the container, and the test strips are arranged so that when the container has a predetermined amount of fluid sample, it can be tilted until the fluid sample contacts the sample receiving portions of the test strips.

### FIELD OF THE INVENTION

The invention relates to a test kit for multi-drug testing and to a method therefor.

### BACKGROUND OF THE INVENTION

The increased availability and use of drugs of abuse by the general population has caused employers, governmental agencies, sports groups and other organizations to utilize drug screening as a condition of employment and/or in order to maintain safety in the work place. Typical drug screening tests are performed for the purpose of quickly identifying on a qualitative basis the presence of drugs in a sample of body fluid, e.g., urine, blood, sputum, pleural cavity and peritoneal cavity fluids, but most often in a urine sample. If preliminary screening results are positive, a complete analysis of the sample may then be carried out in a laboratory. More and more such drug screenings are taking place on site or at the work place and are usually carried out by testing personnel who are not technically trained. It is thus important for the drug screening procedure to be simple yet reliable. Moreover, the test apparatus must enable the testing personnel to avoid all contact with the fluid specimen which is being tested.

The use of test strips that detect specific drugs of abuse have become popular because of their ability to quickly and reliably determine the presence of such drugs. Examples can be found in Gibson U.S. Pat. No. 4,992,296 wherein bibulous paper carriers are impregnated with specific test chemicals for detecting such drug abuse compounds as amphetamine, cocaine, marijuana, and other drugs. Other examples are various commercially available immunoassay test strips. The strips can be used individually or placed in holders of various types for multi-drug testing. A variety of devices have been provided to mount a plurality of test strips to simultaneously immerse them in a sample. See, for example, several American BioMedica Corporation patents such as U.S. Pat. Nos. 5,976,895, 6,372,515, and 6,406,922, and Sagona et al U.S. Pat. No. 6,488,669. The disclosures of each of the foregoing U.S. Pat. Nos. 4,992,296, 5,976,895, 6,372,515, 6,406,922, and 6,488,669 are incorporated herein by reference.

The devices proposed for multi-drug testing using test strips have been somewhat complicated involving the provision of a slotted card to hold the test strips and an inner closure insert to retain the card, thereby adding to the costs of manufacture as well as adding to the costs of technician time to assemble and handle the components.

### BRIEF SUMMARY OF THE INVENTION

The present invention overcomes the foregoing deficiencies by retaining one or more drug of abuse test strips on the underside of a closure cap for a fluid sample container. Each strip has a bottom end defining a sample receiving portion and an indicator spaced longitudinally therefrom to visually indicate the presence of a selected drug of abuse. The closure cap is formed to seal the container, and the test strips are arranged so that when the container has a predetermined amount of a fluid sample, it can be tilted until the fluid sample contacts the receiving portions of the test strips.

More particularly, the undersurface of the closure cap is formed with a plurality of longitudinally extending slots conforming to the longitudinal shapes of the test strips and in which the test strips are retained so that when the sample container is tilted in the direction of the bottom ends of the test strips, fluid sample will contact the sample receiving portions of the test strips.

The container is preferably substantially transparent or translucent. Also, preferably, one or more projections are provided extending laterally outwardly from a location on the container adjacent the sample receiving portions of the test strips to serve as feet to support the container in a tilted position that restricts contact of the fluid sample to the sample receiving portions of the test strips.

### BRIEF DESCRIPTION OF THE DRAWINGS

For a more complete understanding of the present invention, reference is now made to the following descriptions taken in conjunction with the accompanying drawing, in which:

FIG. 1 is a perspective, exploded view of the test kit of the invention, showing the sample container and closure cap with longitudinally extending slots formed to retain a plurality of drug of abuse test strips;

FIG. 2 is a perspective view of the test kit of FIG. 1 with a fluid sample in the container, omitting fluid level markings, and with the closure cap sealing the container;

FIG. 3 shows the closed sample container in an upright position in shadow and supported in a tilted position by a laterally outwardly extending closure cap projection; and

FIG. 4 is a cross-sectional view taken on line 4-4 of FIG. 3 in the direction of the arrows, showing the fluid sample contacting the sample receiving portions of a test strip.

### DETAILED DESCRIPTION OF THE INVENTION

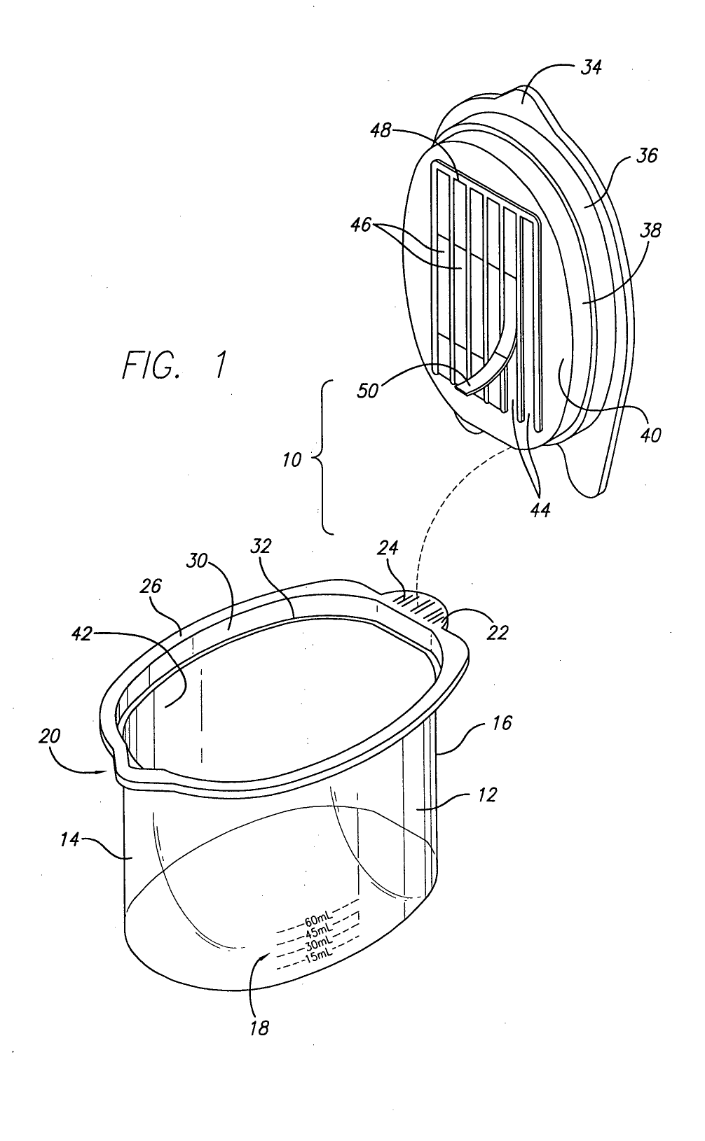

Referring to FIG. 1, a test kit of the invention, indicated generally at 10, includes a cup-like container 12 formed of substantially transparent or translucent plastic, such as polypropylene homopolymer of a type often used for collecting urine specimens. In a particular embodiment, the container 12 has an oblong wall with rounded front and rear ends, respectively 14 and 16, and is formed with markings 18 indicating various volume amounts. In a particular embodiment, a single marking can be used indicating a predetermined amount of fluid to be poured into or left in the container 12. The container can optionally be formed with a pour spout 20 on top of the front end 14 of the container 12 and with a short thumb and forefinger handle 22 on top of the rear end 16 of the container 12 having a plurality of gripping ridges 24 formed into its top surface. Otherwise, the top edge 24 of the container 12 is preferably formed flat to provide a mating surface for a closure cap 28 as described below. A top margin 30 of the container 12 forms an internal ridge 32.

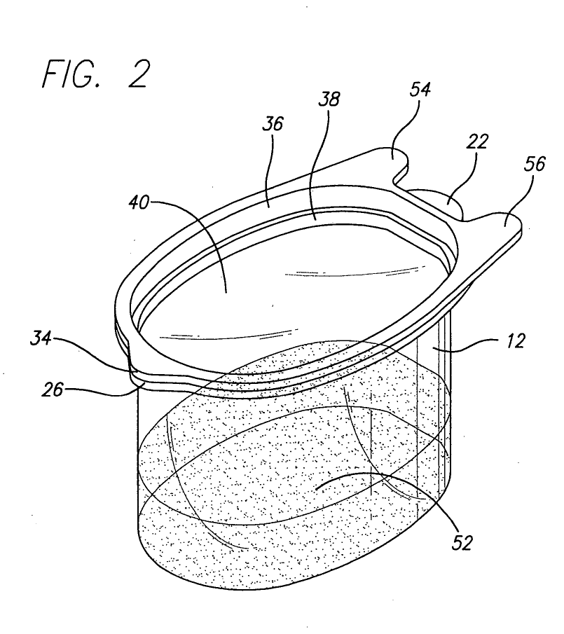

Referring additionally to FIG. 2, the closure cap 28 is formed to seal the container 12. It has a lip 34 that is substantially coterminous with the top edge 26 of the container 12 (but can be larger) and is also formed flat so as to seat with the top edge 26. A margin 36 of the closure cap 28 is contained by and fits closely to the margin 30 of the container 12 and on the ridge 32. The closure cap 12 is formed with an oblong side wall 38 that depends from the cap margin 36 and terminates in a bottom wall 40. The cap side wall 38 is contained by, and fits closely to, the inner surface 42 of the container 12. The close fit of the cap margin 36 to the container margin 30 and of the cap side wall 38 and container inner surface 42 enables the cap 28 to seal the container 12.

Referring again more particularly to FIG. 1, the undersurface of the cap bottom wall 40 is formed with a plurality of raised longitudinally extending slots 44. In this embodiment, there are six slots 44, but there can be as few as one or as many as eight (or more with a larger container), and preferably, there are 4-6 slots 44. A drug of abuse test strip 46 is retained in each slot, but the kit 10 can also be used with less test strips, even with only one test strip, depending on the drug or drugs of abuse being tested for. The slots 44 have a common top ridge 48 closing the top end of each of the slots. While the bottom ends of the slots can also be closed, it is preferred that the bottom ends of the slots 44 be open.

The drug of abuse test strips 46 each have a bottom end 50 defining a sample receiving portion, which can be in the form of a bibulous material, providing a wicking action to draw in the sample so that it rises along the length of the test strip. An indicator is spaced longitudinally upwardly from the sample receiving portion to visually indicate the presence of a selected drug of abuse. Such an indicator can be one or more selected chemicals that are capable of indicating the presence of a group of drugs, or it can be an immunoassay material that is more selective. The particular nature of the test strip is not per se part of the invention as one can use any of a number of commercially available test strips. For example, the test strips 46 can be of the type made by Bionike of South San Francisco, Calif., Pharmatech of San Diego, Calif., and Arista Biological of Bethlehem, Pa. Such test strips are characterized as immunoassay strips and employ colloidal gold chemistry. The strips can be constructed so that a control color line against a white background emerges when contacted with the sample. The control line is above a test line containing the immunoassay chemistry and confirms that the strip has been sufficiently penetrated by the sample. Accordingly, the presence of a single color line provides a negative indication of the presence of a particular drug. Two color lines provide a positive indication of the presence of a particular drug of abuse. The test strips 46 will generally be labeled with an abbreviation of the drug of abuse for which it indicates, e.g., with COC for cocaine, MOR for morphine, AMP for amphetamine, THC for marijuana (tetrahydrocannibinol), HER for heroin, etc.

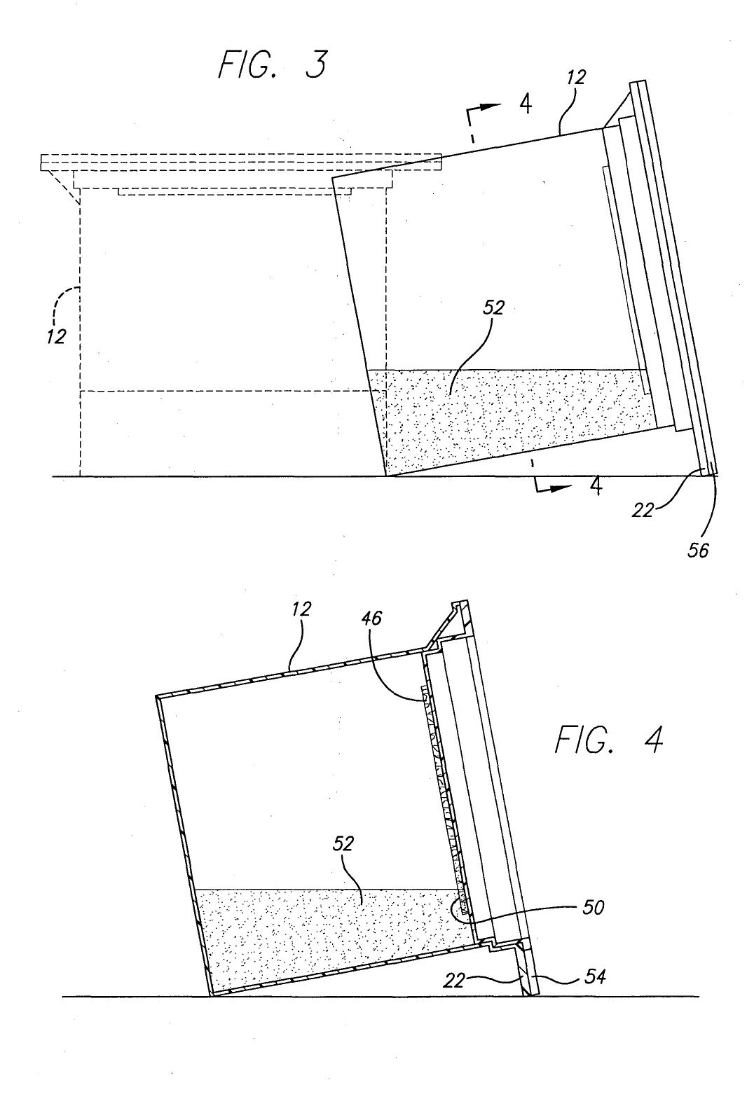

Referring again to FIG. 2, a fluid specimen 52 is placed in the container 12 to a predetermined level, as described further below. The closure cap 28 with, in this case, six test strips 46 retained in the slots 44 on the undersurface of the closure cap 28, positioned over the container 12 and pushed downwardly to seal the container 12. The closure cap 28 is formed with a pair of projections 54 and 56 extending laterally outwardly from a location on the container 12 adjacent the bottom ends 50, i.e., sample receiving portions, of the test strips 46, so as to serve as feet to support the container in a tilted position, as shown in FIG. 3.

Referring to FIG. 3, the closed sample container 12 is shown in an upright position in shadow, and is also shown supported in a tilted position by the projections (one of which, 56, is shown). The projections 54 and 56 are extensions of the closure cap 28, but they could alternatively be formed as extensions from the top edge of the sample container 12. FIG. 4 shows a cross-sectional of the container in its tilted position, and more clearly shows a test strip 46 with its bottom end 50 immersed in the fluid sample. The amount of sample fluid 52 will be seen to be such that it contacts the sample receiving portion 50 of the test strips 46 when the container 12 is tilted to be supported on the feet projections 54 and 56, but not so that it extends substantially up the test strips 46 to or past the drug of abuse indicator.

A devise and method are thus provided for testing a fluid sample. such as a urine specimen. for one or more drugs of abuse. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments described in the specification. As one of ordinary skill in the art will readily appreciate from the disclosure of the present invention, means and methods, or steps, presently existing or later to be developed that perform substantially the same function or achieve substantially the same result as the corresponding embodiments described herein may be utilized according to the present invention. Accordingly, the appended claims are intended to include within their scope such means, methods, and steps.

1. A test kit for testing a fluid sample for at least one drug of abuse using at least one drug of abuse test strip having a bottom end defining a sample receiving portion and having an indicator spaced longitudinally therefrom to visually indicate the presence of a selected drug of abuse, comprising:
a cup-like sample container having an open top end, for containing a predetermined amount of fluid sample to be tested;
a closure cap positionable over the open top end of the container, formed to seal the container; and
structure on the undersurface of the closure cap for retaining said test strip so that when the sample container is tilted in the direction of the bottom end of the test strip, fluid sample will contact the sample receiving portion of the test strip.

2. The test kit of claim 1 including at least one projection on the closure cap or container extending laterally outwardly from a location on the container adjacent the sample receiving portion of the test strip to define a tilt stop for the container to limit the extent of tilting of the container whereby to restrict contact of the fluid sample to said sample receiving test strip portion.

3. The test kit of claim 2 in which there are two of said projections serving as feet to support the container in its tilted position.

4. The test kit of claim 2 in which said projection is on the closure cap.

5. The test kit of claim 4 in which the open top end of the cup-like container is oblong with rounded ends, said projections extending from opposite sides of one of said rounded ends.

6. The test kit of claim 1 including said drug of abuse test strip in said undersurface structure.

7. The test kit of claim 1 in which said undersurface structure comprises at least one longitudinally extending slot in the undersurface of the cap conforming to the longitudinal shape of the test strip and in which the test strip can be retained.

8. The test kit of claim 7 in which the slot has a closed top end and an open bottom end.

9. The test kit of claim 1 in which the structure on the undersurface of the closure cap is formed to retain a plurality of drug of abuse test strips, each having a bottom end defining a sample receiving portion and having an indicator spaced longitudinally therefrom to visually indicate the presence of a selected drug of abuse.

10. The test kit of claim 8 in which the structure on the undersurface of the closure cap is formed to retain from 4 to 6 of said drug of abuse test strips.

11. The test kit of claim 1 in which the container is substantially transparent or translucent.

12. A test kit for testing a fluid sample for drugs of abuse, comprising:
a plurality of drug of abuse test strips, each having a bottom end defining a sample receiving portion and having an indicator spaced longitudinally therefrom to visually indicate the presence of a selected drug of abuse
a cup-like substantially transparent or translucent sample container having an open top end, for containing a predetermined amount of fluid sample to be tested;
a closure cap positionable over the open top end of the container, formed to seal the container; and
a plurality of longitudinally extending slots in the undersurface of the cap conforming to the longitudinal shapes of the test strips and in which the test strips are retained on the undersurface of the closure cap so that when the sample container is tilted in the direction of the bottom ends of the test strips, fluid sample will contact the sample receiving portions of the test strips;
said closure cap having a pair of projections extending laterally outwardly from a location on the container adjacent the sample receiving portions of the test strips and serving as feet to support the container in a tilted position to define a tilt stop for the container that limits the extent of tilting of the container whereby to restrict contact of the fluid sample to said sample receiving test strip portion.

13. The test kit of claim 12 in which the slots each have a closed top end and an open bottom end.

14. The test kit of claim 12 in which there are from 4 to 6 of said drug of abuse test strips and 4 to 6 of said slots.

15. A method for testing a fluid sample for at least one drug of abuse, comprising:
providing a cup-like sample container having an open top end and containing a predetermined amount of fluid sample to be tested;
retaining on the undersurface of a closure cap at least one drug of abuse test strip having a bottom end defining a sample receiving portion and having an indicator spaced longitudinally therefrom to visually indicate the presence of a selected drug of abuse;
sealing the container with the closure cap; and
tilting said container in the direction of the bottom end of the test strip so that the fluid sample contacts the sample receiving portion of the test strip.

16. The method of claim 15 in which there is at least one projection on the closure cap or container extending laterally outwardly from a location on the container adjacent the sample receiving portion of the test strip to define a tilt stop for the container to limit the extent of tilting of the container whereby to restrict contact of the fluid sample to said sample receiving test strip portion.

17. The method of claim 16 in which there are two of said projections serving as feet to support the container in its tilted position.

18. The method of claim 16 in which said projection is on the closure cap.

19. The method of claim 18 in which the open top end of the cup-like container is oblong with rounded ends, said projections extending from opposite sides of one of said rounded ends.

20. The method of claim 15 in which the test strip is retained by at least a longitudinally extending slot in the undersurface of the cap conforming to the longitudinal shape of the test strip.

21. The method of claim 20 in which the slot has a closed top end and an open bottom end.

22. The method of claim 15 in which a plurality of drug of abuse test strips are retained on the undersurface of the closure cap, each test strip having a bottom end defining a sample receiving portion and having an indicator spaced longitudinally therefrom to visually indicate the presence of a selected drug of abuse.

23. The method of claim 22 in which from 4 to 6 of said drug of abuse test strips are retained on the undersurface of the closure cap.

24. The test kit of claim 15 in which the container is substantially transparent or translucent.

25. A method for testing a fluid sample for drugs of abuse, comprising:
providing a cup-like substantially transparent or translucent sample container having an open top end and containing a predetermined amount of fluid sample to be tested;
retaining on the undersurface of a closure cap a plurality of drug of abuse test strips, each having a bottom end defining a sample receiving portion and having an indicator spaced longitudinally therefrom to visually indicate the presence of a selected drug of abuse, the undersurface of the closure cap having a plurality of longitudinally extending slots conforming to the longitudinal shapes of the test strips and in which the test strips are retained;
sealing the container with the closure cap; and
tilting the container in the direction of the bottom ends of the test strips so that the fluid sample contacts the sample receiving portions of the test strips.

26. The method of claim 25 in which the slots each have a closed top end and an open bottom end.

27. The method of claim 25 in which there are from 4 to 6 of said drug of abuse test strips and 4 to 6 of said slots.
